(19) United States
(12) Patent Application Publication
(10) Pub. No.: US 2006/0034886 A1
(43) Pub. Date: Feb. 16, 2006

(54) Bonded fiber structures for use in controlling fluid flow

(76) Inventors: Bennett C. Ward, Midlothian, VA (US); Jian Xiang, Midlothian, VA (US); David B. Harris, Chester, VA (US); Jackie F. Payne JR., Chester, VA (US)

Correspondence Address: HUNTON & WILLIAMS LLP;Riverfront Plaza, East Tower, 951 E. Byrd Street, Richmond, VA, 23219-4074, US

(21) Appl. No.: 11/185,381

(22) Filed: Jul. 20, 2005

Related U.S. Application Data
(60) Provisional application No. 60/590,463, filed on Jul. 23, 2004.

(51) Int. Cl. A61F 2/00

(52) U.S. Cl. 424/424; 604/246

(57) ABSTRACT

A flow control element is provided for use in selectively controlling the flow of a liquid through an annular conduit. The flow control element comprises a self-sustaining, three dimensional fibrous element comprising a network of polymeric fibers. These fibers are disposed in a highly dispersed and randomly spaced orientation and are bonded to each other at spaced apart points of contact to form a tortuous interstitial passage through the fiber element. The fibrous element has a substantially uniform density and is sized for disposition in the lumen with an interference fit relative to the inner conduit surface. The fibrous element divides the lumen into a proximal lumen portion and a distal lumen portion when so disposed. The fibrous element is adapted to prevent passage of the liquid through the conduit when disposed therein absent a differential pressure between the distal conduit portion and the proximal conduit portion of at least a first predetermined critical differential pressure. The fibrous element allows passage of the liquid through the conduit when the differential pressure between the distal conduit portion and the proximal conduit portion equals or exceeds the first predetermined critical differential pressure. In some embodiments, the fibrous element may also be adapted so that it will move from its first position in the lumen to a second position when the differential pressure between the distal conduit portion and the proximal conduit portion equals or exceeds a second predetermined critical differential pressure.

### CROSS-REFERENCE TO RELATED APPLICATIONS

This application claims priority to U.S. Provisional Application No. 60/590,463 filed Jul. 23, 2004, which is incorporated herein by reference in its entirety.

### BACKGROUND OF THE INVENTION

The invention relates generally to bonded, polymeric fiber structures and, more particularly, to bonded polymeric fiber structures adapted for use as fluid flow controllers in devices such as those used to selectively deliver oral medication to a patient.

In the field of medicine, drug delivery devices comprised of a conduit (for example, a common drinking straw) and an accompanying filter device are commonly employed to deliver a single dose of a drug to a patient. The filters, which are disposed within the straw or other conduit, are used as a substrate to support a dose of medication (for example, in powdered form). In use, the patient draws a fluid through the straw, and consequently, through and/or around the filter supporting the medication dose. The medication is solublized or suspended in the liquid so that the patient can ingest the medication with the fluid.

U.S. Pat. Nos. 5,780,058; 5,985,324; and 5,985,324 to Wong et al. disclose several such medication delivery devices. These devices comprise a tube or straw in which a plug or controller element is placed. The plug or controller acts as a one-way valve that allows fluid to flow through or around the plug or controller as long as suction is applied to the downstream side of the plug or controller. Depending on its configuration, the plug or controller may be formed from porous or non-porous material. In one embodiment disclosed in the Wong '324 patent, a controller is formed from relatively large (0.25 in. to 0.35 in. diameter) fibers bonded together to form a cylinder.

U.S. Pat. No. 6,217,545 to Haldopoulous discloses another medication delivery device. Haldopoulous describes a medication delivery device comprising a straw and a filter disposed within the straw. The Haldopoulous filter is constructed to have at least two distinct regions, a central core region having a more dense construction and an outer peripheral region having a less dense construction. The Haldopoulous filter is constructed so that frictional forces in the outer peripheral region maintain the filter in place when it is under static conditions. When liquid traveling through the conduit comes in contact with the Haldopoulous filter, the force of the liquid on the filter overcomes the frictional forces holding the filter in place and causes the filter to move. The fibers in the filter are generally aligned with the axis of the straw. The Haldopoulous filter may have problems that result from its complexity and the inherent variability of a multi-density construction.

The fiber-based filter devices disclosed in the above patents suffer from a number of disadvantages. For example, when the disclosed fiber-based filters are contacted by a fluid, the fluid tends to wet out the fibers and may be drawn into and through the filter before suction is applied on the opposite side of the filter. As a result, the medication may be wetted before delivery is initiated.

Another problem with existing devices is that they may require a high degree of suction (differential pressure) in order to draw liquid through the filter. This can make the device unusable by small children or the elderly. Previous fiber-based filters also suffer from a lack of uniformity and consistent response to an applied differential pressure. They are also difficult to tailor to different device sizes and differential pressure requirements.

Yet another problem with the previously disclosed fiber-based devices is that the fibers used typically have a finish on the fiber surface that may change over time or have unanticipated reactions with the medications to be delivered using the device. In some cases, special finishes that comply with food and drug regulations for safety for food contact may be required.

The present invention overcomes these problems and offers improvements over known devices and assemblies in the art. Although certain deficiencies in the related art are described in this background discussion and elsewhere, it will be understood that these deficiencies were not necessarily heretofore recognized or known as deficiencies. Furthermore, it will be understood that, to the extent that one or more of the deficiencies described herein may be found in an embodiment of the claimed invention, the presence of such deficiencies does not detract from the novelty or non-obviousness of the invention or remove the embodiment from the scope of the claimed invention.

### SUMMARY OF THE INVENTION

In one aspect of the invention, a flow control element is provided for use in selectively controlling the flow of a liquid through an annular conduit. The conduit has an inner conduit surface and an inner cross-sectional circumference and defines a lumen extending from a proximal end of the conduit to a distal end of the conduit. The flow control element comprises a self-sustaining, three dimensional fibrous element comprising a network of polymeric fibers. These fibers are disposed in a highly dispersed and randomly spaced orientation and are bonded to each other at spaced apart points of contact to form a tortuous interstitial passage through the fiber element. The fibrous element has a substantially uniform density and is sized for disposition in the lumen with an interference fit relative to the inner conduit surface. When so disposed, the fibrous element divides the lumen into a proximal lumen portion and a distal lumen portion. The fibrous element is adapted to prevent passage of the liquid from the distal lumen portion to the proximal lumen portion absent a differential pressure between the distal conduit portion and the proximal conduit portion of at least a first predetermined critical differential pressure. The fibrous element allows passage of the liquid from the distal lumen portion to the proximal lumen portion when the differential pressure between the distal conduit portion and the proximal conduit portion equals or exceeds the first predetermined critical differential pressure. In some embodiments, the fibrous element may also be adapted so that it will move from its first position in the lumen to a second position when the differential pressure between the distal conduit portion and the proximal conduit portion equals or exceeds a second predetermined critical differential pressure.

### BRIEF DESCRIPTION OF THE DRAWINGS

The present invention can be more fully understood by reading the following detailed description of the presently preferred embodiments together with the accompanying drawings, in which like reference indicators are used to designate like elements, and in which:

FIG. 1 is a perspective view of an illustrative medication delivery device having a fluid control element according to an embodiment of the invention;

FIG. 2 is a sectioned perspective view of a fluid control element according to an embodiment of the invention;

FIG. 3 is a section view of an illustrative sheath-core bicomponent fiber that may be used in fluid control elements according to various embodiments of the invention;

FIG. 4 is a photomicrograph of the bonded fiber structure of a fluid control element according to an embodiment of the invention;

FIG. 5 is a perspective view of a medication delivery device having a fluid control element according to an embodiment of the invention showing a portion of a usage sequence for the medication delivery device;

FIG. 6 is a perspective view of a medication delivery device having a fluid control element according to an embodiment of the invention showing a portion of a usage sequence for the medication delivery device; and

FIG. 7 is a perspective view of a medication delivery device having a fluid control element according to an embodiment of the invention showing a portion of a usage sequence for the medication delivery device.

### DETAILED DESCRIPTION OF THE INVENTION

The present invention provides various embodiments of a flow control element for use in fluid conduits where it is desirable to allow passage of a gas through the conduit while selectively preventing the passage of a liquid having certain characteristics. The flow control elements of the invention are formed as substantially uniform, self-sustaining, bonded fiber structures. The fibers used may have specific characteristics that make the resulting structure resistant to the liquid when the structure is contacted by the liquid. This prevents the liquid from wetting out the fibers or being drawn into the fiber structure. The fiber structures are adapted, however, so that when a predetermined differential pressure is applied across the flow control element, the liquid is drawn into and through the fluid control element.

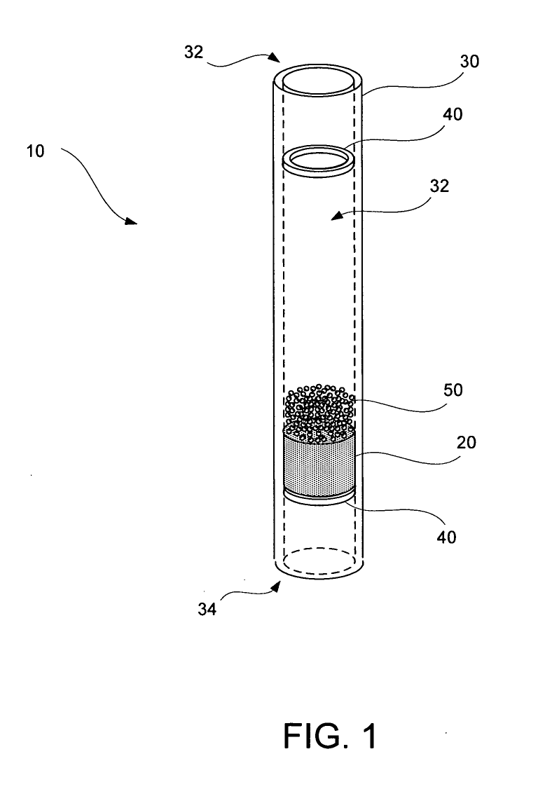

The flow control elements of the invention are well-suited for use in the previously described variety of medication delivery device in which medication is delivered to a patient through a drinking straw. FIG. 1 is a perspective view of an illustrative drug delivery device of this type. As shown in FIG. 1, the drug delivery device 10 includes a flow control element 20 that is disposed in the lumen 32 of a tubular conduit 30 having a proximal end and a distal end. The conduit 30 may be constructed of a transparent or translucent material such that the flow control element 20 is visible from outside the device. The conduit 30 may be in the form of a drinking straw or may be a portion of a syringe or other device for selectively administering a medication. As shown in FIG. 1, a medication 50 in powdered, pellet or other readily dispersible or dissolvable form may be disposed within the lumen 32 proximal to the flow control element 20. Alternatively, the medication 50 may be disposed within the interstices of the bonded fiber structure of the flow control element 20. In either case, the flow control element provides support for the medication 50 and prevents the release of medication out of the distal end of the conduit 30 until in solution with the ingested fluid.

The medication delivery device 10 is structured so that when suction is applied at the proximal end of the conduit 30, a fluid may be drawn into the distal end of the conduit 30. Ideally, gas (e.g., air) passes readily through the flow control device when any differential pressure (ΔP) is applied across the flow control device. Liquid, however, is preferably excluded unless a sufficiently high ΔP is applied. When liquid is introduced to the distal end of the conduit 30 and a sufficiently high ΔP is applied, the liquid passes through and/or around the fluid control element 20 and encounters the medication 50. If the ΔP is maintained, a mixture of liquid and medication is delivered to the proximal end of the conduit 30.

In a typical configuration, the medication delivery device 10 uses a form of drinking straw as the conduit 30. The distal end of the straw is typically immersed in a liquid such as water, juice, etc. so that the user can draw the liquid through the straw and out the straw's proximal end into the user's mouth. In typical usage, the distal end of the straw may be placed into the liquid prior to the actual use by the user. As a result, the liquid may enter into the lumen of the straw before it is required. In order for the device to properly deliver the medication when the user draws on the straw, the liquid must be prevented from contacting the medication.

The present invention provides bonded fiber structures that may be used as flow control elements in the medication delivery device 10 and in other similar applications. These structures are configured so that they allow the passage of air but prevent liquid from passing through or around the flow control element unless or until a predetermined ΔP is applied across the flow control element. The structures may be tailored to meet specific ΔP requirements while maintaining an effective seal against liquid under specified conditions. The structures are also configured so that they prevent the passage of solid drug particles through the distal end of the straw.

Accordingly, a flow control element according to an embodiment of the invention comprises a three dimensional, self-sustaining, network of bonded polymeric fibers. This network defines a tortuous flow path for passage of fluids through the element. The structure, density and material of the fibers may be tailored to provide desired overall element porosity and fiber surface area. The materials and configuration of the fibers also determine the degree to which the fibers attract or repel certain fluids. In particular embodiments, the flow control element comprises fibers that provide a hydrophobic surface that repels water.

The bonded fiber structures of the invention may be formed from a web of thermoplastic fibrous material or fibers. In some embodiments, these fibers are melt-blown sheath-core bicomponent fibers in which the sheath component is formed from a hydrophobic polymer or is treated or coated so as to present a hydrophobic surface. The web may be formed as an interconnecting network of highly dispersed continuous (e.g., filament) and/or discontinuous (e.g., staple) bicomponent fibers bonded to each other at various points of contact to provide a series of tortuous fluid paths with very high surface areas.

It will be understood that the term “bicomponent” is not meant to limit the fibers used in embodiments of the invention to a particular number of components; rather, it is understood that “bicomponent” materials may also be “multi-component” materials having two or more materials.

A cross-section of a typical sheath-core bicomponent fiber 22 is shown in FIG. 3. The sheath-core fiber 22 has a core region 24 comprising one or more polymeric core materials and a sheath region 26 comprising one or more polymeric sheath materials.

Various commonly owned prior art patents clearly show and describe preferred processing techniques and apparatus for producing bicomponent fibers and forming three-dimensional, self-sustaining structures from such fibers. These patents include U.S. Pat. Nos. 5,509,430, 6,026,819, and 6,103,181, each of which is incorporated herein in its entirety.

In a preferred embodiment, a bonded fiber structure for use in a flow control element is made by melt-blowing a plurality of sheath-core bicomponent fibers to form a network of highly dispersed and randomly spaced polymeric fibers. Hot air is used to draw and attenuate the fibers upon extrusion from a melt-blow spin beam, which are then collected and cooled to form a randomly distributed loosely bonded web of fibers. This web may then be drawn through a die heated with hot air or steam to form a porous, bonded fiber rod, which is then cooled and cut to desired lengths.

The above process provides a consistent melt-blown fiber web that produces a substantially homogeneous structure of randomly distributed, non-aligned, fibers bonded to one another at spaced apart points of contact. This structure has a uniform density and porosity throughout and provides flow control elements that are highly regular with repeatable overall porosity.

The fibers used in fluid control elements of the invention may include, but are not limited to melt-blown bicomponent fibers formed from one or more of the group comprising hydrocarbon resins, polyolefins, such as polyethylene, polypropylene, and copolymers thereof; polyesters, such as polyethylene terephthalate, polyethylene terephthalate copolymers and polybutylene terephthalate and copolymers thereof; polyamides, such as nylon 6 and nylon 66 and copolymers thereof; fluoropolymers, polyacrylates, polycarbonates, polyvinyl chloride, polystyrene, ABS, acetal homopolymers and copolymers, polyacrylonitrile, polymethylmethacrylate, polyvinyl alcohol, polyethyleneglycol, ethylene vinyl alcohol copolymers, copolymers of ethylene with ethylene methacrylic acid, ethylene acrylic acid, ethylene-vinyl acetate, and ethylene methyl acrylate, and cationic di-polyesters.

Embodiments of the bonded fiber structures of the invention may comprise bonded bicomponent staple fibers, bonded bicomponent filament fibers, and mixtures thereof, that exhibit hydrophobic properties. In the selection of such polymers, as described above, it is preferred that for sheath-core fibers, the sheath component has hydrophobic properties, i.e., is adapted to repel aqueous liquids commonly used in medication delivery.

In preferred embodiments, the sheath material is formed from hydrophobic (i.e., low surface energy) materials such as polyethylene, polypropylene, copolymers of ethylene and methacrylic acid and thermoplastic fluoropolymers. In one preferred embodiment, the bonded fiber structure used in the flow control element comprises melt-blown bicomponent fibers having a core material of polypropylene and a sheath material of ethylene/methacrylic acid copolymer. In another embodiment, the bonded fiber structure comprises a polypropylene core material and a low density polyethylene sheath material.

To provide structural integrity, the fiber core may be formed using any crystalline polymer including but not limited to polyamides (such as nylon 6, nylon 6,6 and other nylons) polyesters (such as polyethylene terephthalate, polybutylene terephthalate, polypropylene terephthalate and polylactic acid) and other polyolefins (such as syndiotactic, isotactic polypropylene and polyethylene). The core polymer need not be hydrophobic. However, the use of a hydrophobic core material may be advantageous to avoid concerns that could arise from incomplete coverage of the core by the sheath material.

A significant advantage of the use of melt-blown fibers is that finishes or lubricating fluids are typically not used to produce them; hence the resulting fibers do not have a residual finish on the surface of the fiber. This ensures that the hydrophobicity of the fiber and the performance characteristics of the bonded structure do not change with time as is the tendency with structures having a degradable finish.

In alternative embodiments the fibers used to produce the bonded fiber structures of the invention may be coated with a hydrophobic material either before or after they are bonded to form the self-supporting bonded fiber structure.

The average maximum cross-sectional dimension (average diameter for fibers with a circular cross-section) of fibers used in the bonded fiber structures of the invention may be in a range of about one micron to about 30 microns. In preferred embodiments, the average maximum cross-sectional fiber dimension is in a range from about 15 microns to about 20 microns. The porosity of the bonded fiber structures of the invention may be in a range of about 70% to about 98%. In preferred embodiments, the porosity of the bonded fiber structures of the invention may be in a range of about 85% to about 92%. As previously noted, the density of the fiber structure is substantially constant throughout the structure.

Although the preferred method of manufacturing fibers for the bonded fiber structures of the invention is using the melt-blowing process described above, it should be appreciated that other methods of manufacturing such filters may alternatively be employed. These methods include continuously spinning by conventional methods (e.g., melt spinning, spun bonding, dry spinning or wet spinning) a plurality of fibers through a plurality of openings in a die, collecting the fibers on a continuously moving surface to form a highly entangled web in the form of a network of highly dispersed, randomly spaced, continuous fibers. The network is gathered, heated, and passed through a forming die to bond the fibers to each other at their points of contact, then cooled to provide a final bonded fiber structure.

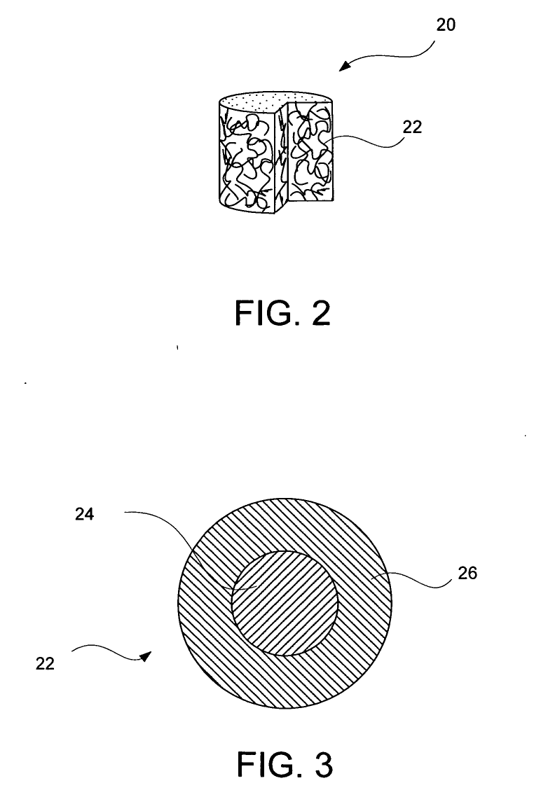

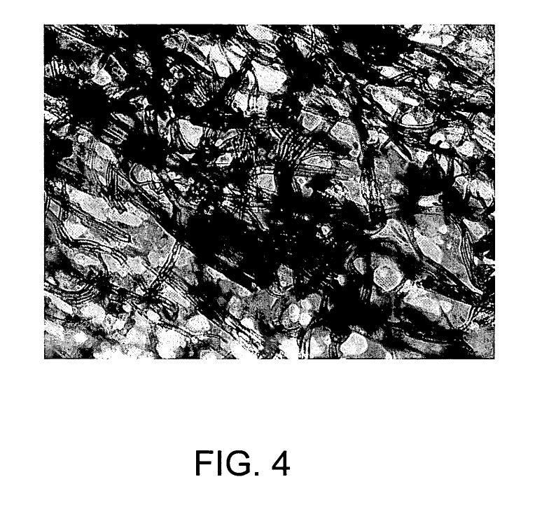

Returning to FIGS. 1 and 2, it can be seen that the flow control element 20 is generally cylindrical in shape. The cross-sectional shape of the fluid control element 20 may be circular, elliptical, polygonal or any other shape required to conform to the interior wall of the conduit 30. As described above, the flow control element comprises a bonded fiber structure formed from a network of highly dispersed and randomly spaced fibers 22. FIG. 4 is a 4× photomicrograph of a bonded fiber structure according to an embodiment of the invention. The photomicrograph is a side view that illustrates the random fiber orientation of the structure.

The flow control element 20 may be fixed in place within the conduit 30 by bonding or other means. In a preferred embodiment, however, the flow control element 20 is adapted to engage the interior sidewalls of conduit 30 by a frictional engagement. In some embodiments, flow control element 20 is constructed so that its circumference is somewhat larger than the inner circumference of the conduit 30, thereby creating an interference fit. As a result, the flow control element 20 will remain fixed in its position until a force is applied that is sufficient to overcome the static friction force between the flow control element 20 and the interior surface of the conduit 30.

In some embodiments, the flow control element 20 must be slightly compressed to conform to the diameter of the conduit 30. The amount of compression required is a factor in the amount of the frictional force between the fluid control element 20 and the inner surface of the conduit 30. In typical embodiments of the invention, an effective ratio of the flow control element diameter (or other maximum dimension for non-circular cross-sections) to the inside diameter of the conduit 30 may be in a range of 1.0 to 1.1. In particular embodiments, this ratio may be in a range from 1.001 to 1.050.

The force required to move the flow control element 20 may be controlled through the tailoring of the various characteristics (e.g., density, porosity, fiber size and fiber material) of the bonded fiber structure and the size of the uninstalled flow control element 20 relative to the conduit 30. Resistance to movement of the flow control element results from the friction force between the flow control element and the inner surface of the conduit 30. The bonded fiber structure may be tailored so that this friction force may be overcome through the application of a sufficiently high ΔP across the flow control element 20. In preferred embodiments, the differential pressure threshold for movement of the flow control element 20 is greater than the differential pressure at which liquid is allowed to pass through or around the flow control element 20.

In particular embodiments, the flow control element 20 may be tailored so that a threshold ΔP for movement of the flow control element 20 is set in a range that allows movement to be initiated by the oral suction applied by a user while drawing fluid into and through the conduit 30. It will be understood that, because movement of the flow control element 20 is resisted by a friction force, the threshold ΔP for movement will be dependent on the friction coefficient between the flow control element 20 and the inner surface of the conduit 30. As noted above, this will depend on the characteristics of the flow control element 20. It will also depend on whether liquid is passing through and, in particular, around the flow control element 20. Because it is generally not desirable to have the flow control element 20 move prior to the establishment of liquid flow therethrough, the flow control element 20 may be adapted to have a relatively high movement threshold ΔP when liquid flow through and around the flow control element has not yet been allowed and a lower movement threshold ΔP when flow through and/or around the flow control element has been allowed.

Referring to FIG. 1, the drug delivery device 10 may be provided with a pair of ribs 40 that serve as limits to the longitudinal travel of the flow control element 20 within the lumen 32. The ribs 40 function to decrease the inner diameter of the lumen 32 at opposite ends and thus prevent movement of the flow control element 20 within the conduit 30 beyond a predetermined distance. It should be appreciated that ribs 40 may be formed as a single unitary piece with conduit 30, or may be separate components affixed to the interior sidewalls of conduit 30. Other mechanisms for limiting the travel of the flow control element 20 may also be used. For example, any mechanism for significantly increasing the friction force between the flow control element and the inner surface of the conduit 30 may be used. This may include tapering the conduit 30, roughening the inner surface of the conduit 30 or providing other forms of projections extending inwardly from the inner surface of the conduit 30. The flow control element 20 is preferably formed with a high enough compression modulus that it will be prevented from deforming under operating differential pressure levels (e.g., the differential pressure established by a patient sucking on the proximal end of the conduit 30) and slipping past the proximal rib 40 or other mechanism for limiting the travel of the flow control element 20 at the proximal end of the conduit 30.

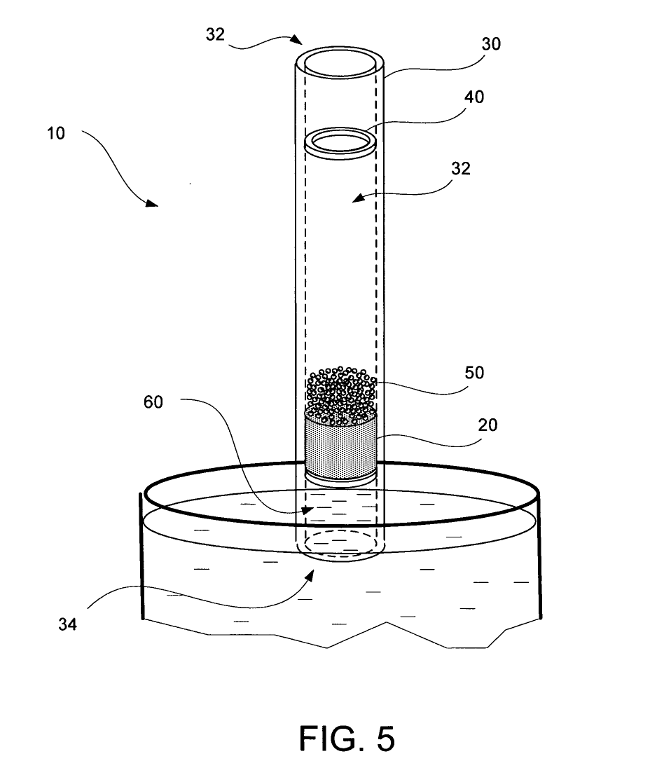

The movement of liquid through and/or around the flow control element 20 and the movement of the flow control element 20 of an illustrative embodiment will now be discussed in more detail. In this embodiment, the flow control element is configured so that a first predetermined critical ΔP is required to draw a particular liquid through and/or around the flow control element 20. A second predetermined critical ΔP, which is greater than or equal to the first predetermined critical ΔP, is required to move the flow control element 20 once the liquid has been allowed to flow through and/or around the flow control element 20. FIG. 1 illustrates the flow control element 20 at an initial position within the conduit 30. This is the position of the flow control element 20 before the patient attempts to draw liquid up through the conduit 30. As shown in FIG. 5, when the distal end 34 of the conduit 30 is placed in a reservoir of the liquid 60 and suction is applied to the proximal end 32 of the conduit (i.e., the pressure in the lumen proximal to the flow control element 20 is reduced), the liquid 60 is drawn into the lumen 32. It will be understood that the suction may be sufficient to draw the liquid 60 into contact with the flow control element 20 but insufficient to draw the liquid through the flow control element 20; i.e., the first predetermined critical ΔP is not reached. Under such circumstances, the hydrophobic nature of the bonded fiber structure of the flow control element 20 will prevent the liquid from penetrating into and though the flow control element 20. If sufficient suction is applied, the first predetermined critical ΔP across the flow control element 20 is reached and the liquid 60 is drawn through and/or around the flow control element 20 to an area of the conduit 30 proximal to the flow control element where the liquid 60 forms a mixture 70 of liquid 60 and medication 50.

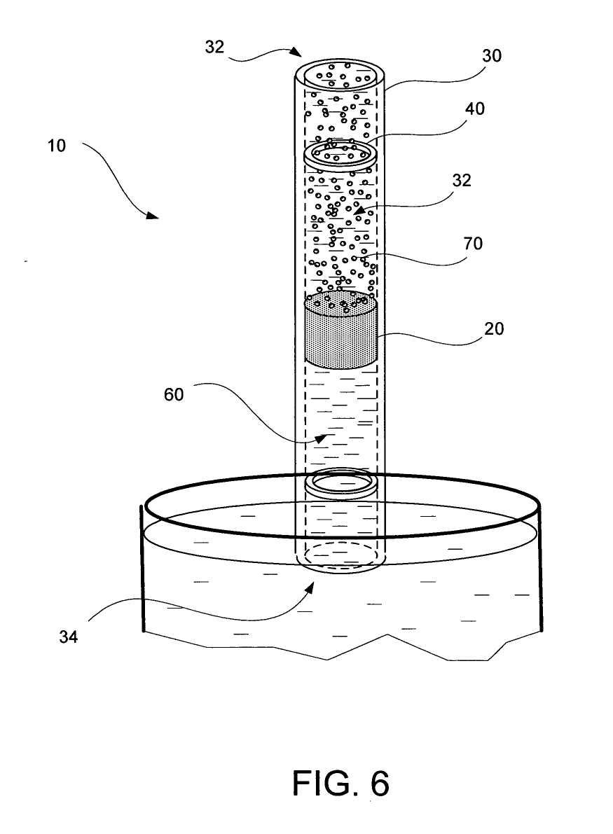

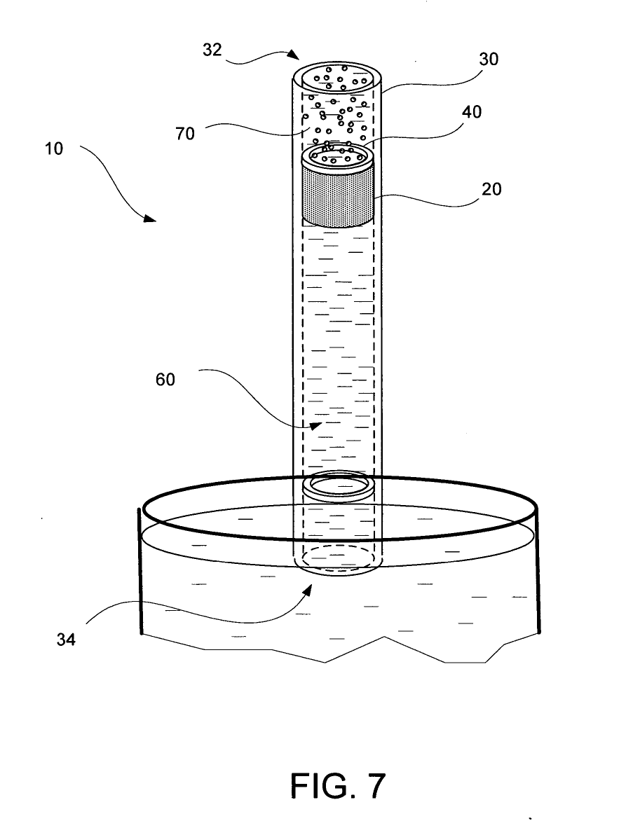

As the liquid 60 passes through and/or around the flow control element 20, the friction force holding the flow control element 20 in place within the conduit 20 is reduced. When the applied suction exceeds a second predetermined critical ΔP, the reduced friction force holding the flow control element 20 in place relative to the conduit 30 is overcome. This causes the flow control element to move toward the proximal end 32 of the conduit 30 as shown in FIG. 6. If sufficient suction is maintained (i.e. the second predetermined critical ΔP is maintained), the flow control element 20 will continue to move in the proximal direction until it reaches the position shown in FIG. 7 where the rib 40 (or other limiting device) prevents further movement.

In most cases, the differential pressure required to make the flow control element 20 move (i.e., the second predetermined ΔP) will be greater than the differential pressure required for liquid flow-through (i.e., the first critical ΔP). This assures that liquid will flow through or around the flow control element to mix with the medication before the flow control element 20 moves. However, in some embodiments, the second critical ΔP may be made approximately equal to the first critical ΔP because the flow control element 20 is not allowed to move under that differential pressure until the friction force is reduced by liquid flow through and/or around the flow control element 20.

As noted above, other mechanisms for limiting the travel of the flow control element 20 may be used. In some embodiments, the travel of the flow control element 20 may be limited at only one end of the conduit 30. For example, a rib 40 (or taper or other limiting mechanism) may be positioned near the proximal end 32 of the conduit 30 but not at the distal end 34.

In use, a patient may be provided a medication delivery device 10 containing a flow control element 20 which supports a single dose of a medication in powder form or in small particles (or a liquid medicine that has been dried to a soluble coating on the fibers). Providing medication in these forms is often advantageous because it enables the drug to be rapidly absorbed in the alimentary canal. In a manner similar to that discussed above, the patient immerses the lower end of the conduit 30 into an ingestible liquid, such as water or juice, and then draws the liquid through the flow control element 20 and the conduit 30 into his or her mouth. When the liquid contacts the medication, the medication is suspended or dissolved into the liquid. As the liquid moves through flow control element 20, it also moves the flow control element 20 toward the proximal end of the conduit 30 and is retained at its final position when the patient stops applying suction. The high flow rate into the alimentary canal using the straw-like conduit allows the administration of medication with minimal perception by the patient and takes advantage of the natural swallowing reflex. The foregoing application provides particular advantage for the oral administration of medication to both pediatric and geriatric patients, especially when the medication is unpalatable.

As discussed above, the bonded fiber structures used in the flow control elements of the invention may be specifically tailored to achieve certain performance goals in a medication delivery device application. These goals may include (1) the prevention of liquid passage prior to the application of a first predetermined critical ΔP across the flow control element; (2) the passage of liquid through the flow control element upon application of a ΔP greater than or equal to the first predetermined critical ΔP; and (3) proximal movement of the flow control element upon application of a ΔP greater than or equal to the second predetermined critical ΔP that is greater than or equal to the first predetermined critical ΔP.

The design levels of the critical ΔPs may vary depending on the intended use of the medication delivery device. For example, it may be advantageous to have relatively low first and second critical ΔPs in devices intended for use by children or the elderly. The flow control elements of the present invention, regardless of size, may be tailored to provide first predetermined critical ΔP levels in a range from about 1 mbar to about 50 mbar. In a particular embodiment of the invention, a flow control element may be tailored to provide a first critical ΔP in a range of about 15 mbar to about 25 mbar.

The flow control element may be further tailored to provide a second critical ΔP in any range of differential pressure greater than or equal to the first critical ΔP. In particular, the flow control element may be tailored so that the second critical ΔP is at a level that is sufficient to establish a particular flow rate of the liquid through the flow control element prior to movement of the flow control element. Tailoring the flow control element in this manner can be used to assure that sufficient fluid has passed through the flow control element to suspend or dissolve medication disposed proximal to the flow control element before the flow control element begins to move in the proximal direction.

The design goals recited above have been met through the use of a combination of material selection and tailoring of the fiber characteristics and geometry of the bonded fiber structure. Testing was conducted to establish the ability to tailor to meet specific quantitative requirements and to establish sensitivity of flow control element performance parameters to changes in, for example, fiber diameter, bonded fiber structure density/porosity and fiber surface energy.

Two exemplary fiber material configurations are presented to demonstrate the ability to tailor a flow control element to specific requirements. The first of these (Fiber 1) uses melt-blown sheath-core bicomponent fibers having a core material of Atofina 3860X polypropylene and a sheath material of Nucrel® 699 ethylene/methacrylic acid copolymer. The second (Fiber 2) uses melt-blown sheath-core bicomponent fibers having the same Atofina 3860X polypropylene core material but with Equistar NA 270 polyethylene as the sheath material. In both cases, the fibers were formed with a 30:70 ratio of sheath material to core material. Both of these fibers have inherently hydrophobic surface materials and both provide well-bonded self-supporting three dimensional structures.

The following paragraphs describe tests conducted to demonstrate the performance of the flow control elements of the invention and to establish performance sensitivity.

A liquid exclusion test procedure was used to establish the ability of the flow control elements to prevent water from penetrating the flow control elements under head pressures of interest. In these tests, flow control elements were positioned 10 mm from the distal end of a straw. The distal end of the straw was then lowered into a reservoir of blue-tinted water tinted with blue food coloring solution to depths sufficient to produce a head pressure of 5 mbar. The straw was held in place for 15 minutes. Successful exclusion criteria were established as no trace of blue-tinted water being observed in, on the sides or on the proximal end of the filter.

Contact angle tests were conducted as a general indicator of hydrophobicity. Contact angle is one convenient way to quantifying the behavior of liquids in contact with solids by measuring the angle formed at the three phase boundary where a liquid, gas and solid intersect. Typically, a contact angle greater than or equal to 90 degrees, indicates that the solid has a low surface energy (i.e., is hydrophobic). On the other hand, a contact angle approaching zero indicates the solid has a high surface energy (i.e., is hydrophilic), which means the liquid has a high affinity to the surface material. Contact angles in between zero and 90 degrees indicate intermediate degrees of hydrophobicity. Tests were conducted on flow control elements of the invention to establish water contact angles for the fiber materials used. The test procedures were conducted using standard test procedures and First Ten Angstroms (FTA) equipment and software.

A water passage test was used to determine the force or vacuum pressure required to deliver water through the flow control element at a rate of 10 mL/min. The test articles were constructed by installing a flow control element in a typical drinking straw having an inside diameter of about 7.2 mm. Tygon® tubing was connected to the straw at both ends. On one side of the straw, the opposite end of the Tygon® tubing was submerged in a beaker of water and on the other side, the opposite end of the tubing was connected to a syringe of a syringe pump. A Validyne digital manometer was connected between the syringe pump and the straw to monitor the pressure. Deionized water was pulled through the filter at a rate of 10 mL/min and the steady state pressure drop was recorded at 50 seconds after starting the test. The data was recorded using a data acquisition software package to confirm steady state conditions were met.

Element movement testing was conducted to demonstrate the movement of the flow control element under various pressure differentials. For these tests, the water passage test set-up was modified so that an assembled straw and flow control element were connected to a piece of Tygon® tubing with a pinch valve operated on a timer. The tubing was then connected to an air filter where the liquid pulled through the straw/flow control element assembly was collected. Downstream of the air filter, a vacuum gauge, flow meter, flow control valve and vacuum pump were installed. The timer was set at 2 seconds and the air flow at 5 L/min. Differential pressures of 50, 75, and 100 mbar were established via the vacuum pump and flow control tube. The volume of liquid pulled through the system and the movements of the filter up the straw were measured. The straw was marked into ten equal increments with 10 at the top and zero at the base. A flow control element that moved half way up the straw was given a score of 5.0 for movement.

The above tests were used to evaluate example flow control elements tailored to meet specific performance requirements. In a particular example, the flow control elements were tailored to a medication delivery device having a polyethylene conduit with an inside diameter of 7.23 mm. For purposes of this example, the medication delivery device has a requirement that the second critical differential pressure be greater than 5 mbar with water as the working liquid. Such a requirement is typical in order to assure that there is no liquid leakage due to the head pressure experienced when the distal end of the conduit is immersed in liquid deep enough so that the flow control element is below the surface of the liquid. By preventing liquid leakage, the flow control element assures that the medication remains dry until a patient applies a suction force that causes the differential pressure across the element to exceed the first critical differential pressure.

A flow control element formed from Fiber 1 was successfully tailored to meet these requirements. This flow control element comprised a bonded fiber structure having a diameter of 7.31 mm, a length of 9.0 mm, an average porosity of 89.0%, and an average fiber diameter of 15.7 microns. Flow control elements of this configuration demonstrated 100% water repellency at a head pressure of at least 5 mbar, initial water flow-through at ΔPs in a range of 15 to 25 mbar and total movement (from bottom to top of the conduit) at vacuum pressure of 100 mbar. The contact angle between water and the flow control element was 128 degrees.

A flow control element formed from Fiber 2 was also successfully tailored to meet the requirements of the proposed medication delivery device. This flow control element comprised a bonded fiber structure having a diameter of 7.30 mm, a length of 9.0 mm, an average fiber size of 15.5 microns, and a porosity 88.1%. This element also demonstrated excellent hydrophobicity by passing the immersion test at the 5 mbar head pressure, permitting initial water flow through at ΔPs in a range of 15 to 25 mbar and exhibiting a contact angle of 119 degrees. This element also successfully moved from the bottom to the top of the conduit at a vacuum pressure of 100 mbar.

The previously described tests were also used to establish the sensitivity of flow control element performance characteristics to fiber characteristics and overall element geometry.

Water exclusion tests were conducted to establish, for a given fiber material (Fiber 1) and element length (9.0 mm), the effect of fiber diameter (ranging from about 10 microns to about 20 microns), porosity of the bonded fiber structure (ranging from about 86% to about 91%) and the ratio of uninstalled flow control element diameter to straw inside diameter (ranging from about 1.001 to about 1.100). Twenty elements were tested for each data point.

The results showed that 100% of the samples tested exhibited acceptable water exclusion at average fiber sizes up to about 18.25 microns. Water exclusion failures began to occur as fiber size was further increased. Similarly, 100% water exclusion performance was achieved for overall fiber structure porosities under 88%. Water exclusion failures began to occur as porosity was raised above this level. Diameter ratio was found to be a secondary parameter with respect to water exclusion with 100% exclusion being reached for all ratios at or above 1.02.

The water passage test was used to establish, for a given fiber material (Fiber 1), the effect of fiber diameter (ranging from about 12 microns to about 20 microns) and bonded fiber structural porosity (ranging from about 85% to about 91%). Results indicated that the differential pressure (i.e., suction) required to deliver water through the flow control element was inversely proportional to fiber size and element porosity. Overall, the required differential pressure ranged from about 14.5 mbar to about 22.9 mbar.

Element movement testing was conducted to establish the effects of element diameter ratio (i.e., ratio of the diameter of the uninstalled flow control element to the inside diameter of the straw) and density of the bonded fiber structure on movement of the flow control element under varying levels of differential pressure. The results showed that movement of the flow control element is more strongly influenced by the element diameter ratio than the density of the flow control element. At the 50 mbar pressure level, for example, a diameter ratio of 1.001 produced a movement score of 8.3 while diameter ratio of 1.100 produced a movement score of 0 (i.e., no movement).

Accordingly, the present invention discloses various embodiments of flow control elements comprising bonded fiber structures having a substantially uniform density and random fiber orientation. The fiber structures may comprise melt-blown bicomponent fibers that have material characteristics tailored to repel water or other liquids. In particular, the fiber may be tailored to be hydrophobic so as to repel water and common beverages. The fibers may be sheath-core fibers in which the sheath comprises a low surface energy material that is hydrophobic in the absence of finish. Alternatively, the fiber structures may comprise fibers to which a hydrophobic finish is applied.

It will be understood by those of ordinary skill in the art that the flow control elements of the invention are not limited to use in medication delivery devices. These elements may be used in any application requiring selective liquid flow control based on differential pressure. It will also be understood that the flow control elements of the invention may be scaled to any size depending on the overall flow requirements.

While the foregoing description includes details and specificities, it is to be understood that these have been included for purposes of explanation only, and are not to be interpreted as limitations of the present invention. Modifications to the embodiments described above can be made without departing from the spirit and scope of the invention, which is intended to be encompassed by the following claims and their legal equivalents.

What is claimed is:

1. A flow control element for use in selectively controlling the flow of a liquid through an annular conduit having an inner conduit surface defining a lumen extending from a proximal end of the conduit to a distal end of the conduit, the flow control element comprising:
a self-sustaining, three dimensional fibrous element comprising a network of polymeric fibers disposed in a highly dispersed and randomly spaced orientation and bonded to each other at spaced apart points of contact to form a tortuous interstitial passage therethrough, said fibrous element
being sized for disposition in said lumen with an interference fit relative to the inner conduit surface, the fibrous element dividing the lumen into a proximal lumen portion and a distal lumen portion when so disposed,
having a substantially uniform density, and
being adapted to prevent passage of the liquid from the distal lumen portion to the proximal lumen portion absent a differential pressure between the distal conduit portion and the proximal conduit portion of at least a first predetermined critical differential pressure, and to allow passage of the liquid from the distal lumen portion to the proximal lumen portion when the differential pressure between the distal conduit portion and the proximal conduit portion equals or exceeds the first predetermined critical differential pressure.

2. A flow control element according to claim 1 wherein the fibrous element is initially disposable within the lumen at a first position and is movable to a second position that is proximal to the first position and wherein the fibrous element is adapted so that movement between the first position and the second position can occur only when the differential pressure between the distal conduit portion and the proximal conduit portion equals or exceeds a second predetermined critical differential pressure that is greater than the first predetermined critical differential pressure.

3. A flow control element according to claim 2 wherein the fibrous element resists movement within the lumen by virtue of a frictional engagement with the inner conduit surface, the frictional engagement providing a static friction force that prevents movement unless the differential pressure between the proximal conduit portion and the distal conduit portion equals or exceeds the second predetermined critical differential pressure and the liquid is in contact with the fibrous element.

4. A flow control element according to claim 1 wherein the first critical differential pressure is in a range of about 1 mbar to about 50 mbar.

5. A flow control element according to claim 1 wherein the first critical differential pressure is in a range of about 15 mbar to about 25 mbar.

6. A flow control element according to claim 1 wherein the polymeric fibers comprise a hydrophobic surface material.

7. A flow control element according to claim 1 wherein the polymeric fibers are melt-blown sheath-core bicomponent fibers comprising at least one sheath material and at least one core material.

8. A flow control element according to claim 7 wherein the at least one sheath material is hydrophobic.

9. A flow control element according to claim 7 wherein the at least one sheath material comprises at least one of the set consisting of polyethylene and ethylene/methacrylic acid copolymer.

10. A flow control element according to claim 7 wherein the at least one core material comprises at least one of the set consisting of nylon 6, nylon 6,6, polyethylene terephthalate, polybutylene terephthalate, polypropylene terephthalate, polylactic acid, syndiotactic polypropylene, isotactic polypropylene and polyethylene.

11. A flow control element according to claim 1 wherein the polymeric fibers have an average maximum cross-sectional dimension in a range of about 1 micron to about 30 microns.

12. A flow control element according to claim 1 wherein the polymeric fibers have an average maximum cross-sectional dimension in a range of about 15 microns to about 20 microns.

13. A flow control element according to claim 1 wherein the conduit surface has a circular cross-section defining a lumen diameter and the fibrous element has a circular cross-section defining a fibrous element diameter and wherein a ratio of the fibrous element diameter to the lumen diameter is in a range of 1.0 to 1.1.

14. A flow control element according to claim 13 wherein the ratio of the fibrous element diameter to the lumen diameter is in a range of 1.001 to 1.050.

15. A flow control element according to claim 1 wherein the fibrous element has a porosity in a range of about 70% to about 98%.

16. A flow control element according to claim 1 wherein the fibrous element has a porosity in a range of about 85% to about 92%.

17. A flow control element according to claim 1 wherein the annular conduit is a portion of a medication delivery device and the fibrous element is adapted for supporting a medication within the lumen, the medication being allowed to mix with the liquid when the liquid passes through the fibrous element.

18. A flow control element for selectively controlling the flow of a liquid through a conduit of a medication delivery device, the conduit having an inner conduit surface defining a lumen extending from a proximal end of the conduit to a distal end of the conduit, the medication delivery device being adapted for selectively delivering a dose of a medication disposed in the lumen to a patient by virtue of the patient applying suction to the proximal conduit end to draw liquid from a reservoir at the distal conduit end through the lumen for mixture with the medication and out through the proximal conduit end, the flow control element comprising:
a self-sustaining, three dimensional fibrous element comprising a network of polymeric fibers disposed in a highly dispersed and randomly spaced orientation and bonded to each other at spaced apart points of contact to form a tortuous interstitial passage therethrough, said fibrous element
being sized for disposition in said lumen with an interference fit relative to the inner conduit surface, the fibrous element dividing the lumen into a proximal lumen portion and a distal lumen portion when so disposed,
having a substantially uniform density,
being adapted to prevent passage of the liquid from the distal lumen portion to the proximal lumen portion absent a differential pressure between the distal conduit portion and the proximal conduit portion of at least a first predetermined critical differential pressure, and to allow passage of the liquid from the distal lumen portion to the proximal lumen portion when the differential pressure between the distal conduit portion and the proximal conduit portion equals or exceeds the first predetermined critical differential pressure.

19. A flow control element according to claim 18 wherein the fibrous element is initially disposable within the lumen at a first position and is movable to a second position that is proximal to the first position and wherein the fibrous element is adapted so that movement between the first position and the second position can occur only when the differential pressure between the distal conduit portion and the proximal conduit portion equals or exceeds a second predetermined critical differential pressure that is greater than the first predetermined critical differential pressure.

20. A flow control element according to claim 19 wherein the fibrous element resists movement within the lumen by virtue of a frictional engagement with the inner conduit surface, the frictional engagement providing a static friction force that prevents movement unless the differential pressure between the proximal conduit portion and the distal conduit portion equals or exceeds the second predetermined critical differential pressure and the liquid is in contact with the fibrous element.

21. A flow control element according to claim 18 wherein the differential pressure between the distal conduit portion and the proximal conduit portion is established by a suction force applied by the patient and wherein the first predetermined critical differential pressure is a function of patient characteristics.

22. A flow control element according to claim 18 wherein the first critical differential pressure is in a range of about 1 mbar to about 50 mbar.

23. A flow control element according to claim 18 wherein the first critical differential pressure is in a range of about 15 mbar to about 25 mbar.

24. A flow control element according to claim 18 wherein the polymeric fibers comprise a hydrophobic surface material.

25. A flow control element according to claim 18 wherein the polymeric fibers are melt-blown sheath-core bicomponent fibers comprising at least one sheath material and at least one core material.

26. A flow control element according to claim 25 wherein the at least one sheath material is hydrophobic.

27. A flow control element according to claim 25 wherein the at least one sheath material comprises at least one of the set consisting of polyethylene and ethylene/methacrylic acid copolymer.

28. A flow control element according to claim 25 wherein the at least one core material comprises at least one of the set consisting of nylon 6, nylon 6,6, polyethylene terephthalate, polybutylene terephthalate, polypropylene terephthalate, polylactic acid, syndiotactic polypropylene, isotactic polypropylene and polyethylene.

29. A flow control element according to claim 18 wherein the polymeric fibers have an average maximum cross-sectional dimension in a range of about 1 micron to about 30 microns.

30. A flow control element according to claim 18 wherein the polymeric fibers have an average maximum cross-sectional dimension in a range of about 15 microns to about 20 microns.

31. A flow control element according to claim 18 wherein the conduit surface has a circular cross-section defining a lumen diameter and the fibrous element has a circular cross-section defining a fibrous element diameter and wherein a ratio of the fibrous element diameter to the lumen diameter is in a range of 1.0 to 1.1.

32. A flow control element according to claim 31 wherein the ratio of the fibrous element diameter to the lumen diameter is in a range of 1.001 to 1.050.

33. A flow control element according to claim 18 wherein the fibrous element has a porosity in a range of about 70% to about 98%.

34. A flow control element according to claim 18 wherein the fibrous element has a porosity in a range of about 85% to about 92%.

35. A flow control element according to claim 18 wherein the fibrous element is adapted for supporting the medication and isolating the medication within the proximal lumen portion.
